(19) United States
(12) Patent Application Publication
(10) Pub. No.: US 2010/0124570 A1
(43) Pub. Date: May 20, 2010

(54) HIGHLY RESILIENT COPOLYMER WITH SHAPE RECOVERY FORCE AND FLEXIBILITY AND THE USE THEREOF FOR THE REPAIR OF ARTICULAR CARTILAGE DEFECTS

(75) Inventors: Soo Hyun KIM, Seoul (KR); Young Mee Jung, Seoul (KR); Sang-Heon Kim, Seoul (KR)

Correspondence Address: OBLON, SPIVAK, MCCLELLAND MAIER & NEUSTADT, L.L.P., 1940 DUKE STREET, ALEXANDRIA, VA, 22314, US

(73) Assignee: KOREA INSTITUTE OF SCIENCE AND TECHNOLOGY, Seongbuk-gu (KR)

(21) Appl. No.: 12/614,919

(22) Filed: Nov. 9, 2009

(30) Foreign Application Priority Data
Nov. 20, 2008 (KR) 10-2008-0115647

(51) Int. Cl. A61K 9/14; C08G 63/08; A61K 35/32; A61P 19/00

(52) U.S. Cl. 424/486; 528/354; 424/93.7

(57) ABSTRACT

The present invention relates to a highly resilient (lactide/glycolide)/ε-caprolactone copolymer with good shape recovery force, flexibility, and biodegradability and a use of such copolymer for the repair of articular cartilage defects. The highly resilient (lactide/glycolide)/ε-caprolactone copolymer of the present invention is capable of rapidly and efficiently inducing cartilage regeneration, can be easily deformed and almost completely restored to its original form after deformation. Further, the highly resilient copolymer of the present invention can be safely and conveniently transplanted to a patient by using an arthroscope without causing economic, physical, and mental burden. Thus, the highly resilient copolymer of the present invention can be effectively used as a polymer scaffold for the repair of cartilage defects.

The present application claims priority from Korean Patent Application No. 10-2008-115647 filed Nov. 20, 2008, the subject matter of which is incorporated herein by reference in its entirety.

### FIELD OF THE INVENTION

The present invention relates to highly resilient (lactide/glycolide)/ε-caprolactone copolymers with good shape recovery force, flexibility, and biodegradability and the use thereof for the repair of articular cartilage defects.

### BACKGROUND OF THE INVENTION

Articular cartilage is the tough, elastic tissue that covers the ends of bones in joint; and enables the bones to move smoothly over one another. When articular cartilage is damaged through injury or lifelong use, it does not heal as rapidly or effectively as other tissues in the body. Instead, the damage tends to spread, allowing the bones to rub directly against each other and resulting in pain and reduced mobility.

Advances in technology and biological engineering are giving new hope to the thousands of peoples who annually experience injuries to the articular cartilage of the knee. Several techniques are now using the patient's own cells and tissues to restore cartilage to weight-bearing cross-sections of bone. Currently, the techniques most widely used clinically for cartilage defects and degeneration include: osteochondral grafting, autologous chondrocyte implantation, and mesenchymal stem cell (MSC) regeneration.

Osteochondral grafting transplants a plug of a bone and healthy cartilage harvested from one area to the defect area. An osteochondral graft can use either the individual's own tissue (autograft) or a matched graft from another source (allograft). If an autograft is planned, the plug of bone and cartilage must come from a non-weight-bearing area that has little contact with other bones, which limits its application to treating smaller lesions. For larger injuries, an allograft is more appropriate, provided that a tissue match can be found or the graft is processed to modify the genetic differences and help prevent rejection.

Autologous chondrocyte implantation is carried out by harvesting healthy cartilage cells, cultivating and implanting them over the defect area. Chondrocytes are mature cartilage cells. In this two-stage surgical procedure, surgeons first use arthroscopic techniques to harvest the cells from a healthy, non-weight-bearing area of the knee joint. The chondrocytes are then treated so they will multiply over several days. During the second surgery, the surgeon cleans the injury site and removes a piece of the soft tissue (periosteum) that covers the tibia. The periosteal tissue is sutured and secured over the injury, and the cultured chondrocytes are then injected beneath the patch. There, the chondrocytes will eventually produce a form of cartilage that is very much like the original articular cartilage.

Because autologous chondrocyte implantation uses the patient's own cells, there is no danger of rejection by the immune system. Complications are rare and, in most cases, the procedure results in a restoration of joint movement without pain. Autologous chondrocyte implantation is not appropriate for every patient. Several factors must be considered in decision making, including the size of the defect, the number and type of previous surgeries, the patient's demands and expectations, the location of the injury, and the presence of coexisting lesions. The patient's age and the reason for cartilage deterioration must also be considered. An older person with advanced osteoarthritis is not a candidate for autologous chondrocyte implantation, but a younger person with a traumatic injury to the knee may be an appropriate candidate.

The newest technique being developed uses mesenchymal stem cells (MSCs). MSCs are relatively undifferentiated, embryonic-like cells with the potential to develop into various types of cells. They are found in adult bone marrow and in the periostcum, a tissue layer over the areas of bone not covered by articular cartilage.

Research suggests that MSCs can be withdrawn from the bone marrow, placed in a gel matrix, and implanted at the defect, where they develop into new cartilage. Research is being conducted on the possibility of placing MSCs in a gel, then inserting the gel into the cartilage defect. Because MSCs appear to be capable of organizing in the same way that cartilage is structured, it is hoped that they will be able to regenerate articular cartilage.

Several studies have been conducted in an effort to overcome the above-mentioned limitations of the previously known therapies for cartilage regeneration. PCT International Patent Publication Nos. WO 1994/20151 and WO 1995/33821, for example, describe the in vivo growth and preparation of cartilage by growing stromal cells, such as chondrocytes, progenitor-chondrocytes, fibroblasts and/or fibroblast-like cells, on a three-dimensional scaffold or framework. U.S. Pat. No. 5,041,138 relates to the neomorphogenesis of cartilage in vivo from cell culture by using a scaffold/cell mixture for the growth and implantation of cartilaginous structures. Korean Patent Publication Nos. 2003-15160, 2005-64068, and 2007-113572 disclose cartilage therapeutic agents using a scaffold/cell mixture or a hydrogel/cell mixture.

However, none of the above patent applications discloses a method which would regenerate diseased cartilage to a functional state.

The present invention is directed to overcoming the above-noted deficiencies in the art.

### SUMMARY OF THE INVENTION

One of the objectives of the present invention is to provide a polymer scaffold useful for the repair of cartilage defects which is capable of effectively inducing cartilage regeneration and can be transplanted to a cartilage defect area of a patient in a simple and convenient way.

In order to achieve the above objective, one embodiment of the present invention relates to a highly resilient (lactide/glycolide)/ε-caprolactone copolymer with good shape recovery force, flexibility, and biodegradability.

Another embodiment of the present invention relates to a polymer scaffold for the repair of cartilage defects which is prepared by using the above highly resilient (lactide/glycolide)/ε-caprolactone copolymer.

Yet another embodiment of the present invention relates to a method of transplanting the above polymer scaffold to a cartilage defect area of a patient by using an arthroscope.

### BRIEF DESCRIPTION OF THE DRAWINGS

The embodiments of the present invention will be described in detail with reference to the following drawings.

FIG. 1A is a schematic illustration of a polymer scaffold made of a (lactide/glycolide)/ε-caprolactone copolymer according to the present invention which has been folded and inserted into an arthroscopc.

FIG. 1B is a schematic illustration of a polymer scaffold made of a (lactide/glycolide)/ε-caprolactone copolymer according to the present invention which has been folded and inserted into a special manufactured arthroscope equipped with a fixation bar.

FIG. 2A is a scanning electron microscope (SEM) photograph of the surface of a polymer scaffold made of a lactide/ε-caprolactone copolymer according to the present invention as described in Example 1.

FIG. 2B is a SEM photograph of the cross-section of a polymer scaffold made of a lactide/ε-caprolactone copolymer according to the present invention as described in Example 1.

FIG. 3A is a series of photographs showing the shape recovery force of a polymer scaffold made of a lactide/ε-caprolactone copolymer according to the present invention as described in Example 1.

FIG. 3B is a series of photographs showing the flexibility of a polymer scaffold made of a lactide/ε-caprolactone copolymer according to the present invention as described in Example 1.

FIG. 4 is a graph showing the restoration rate of a polymer scaffold made of a lactide/ε-caprolactone copolymer according to the present invention as described in Example 1 by measuring the change in length before and after deformation strain is applied thereto.

FIG. 5 is a graph showing the restoration rate of a polymer film made of a (lactide/glycolide)/ε-caprolactone copolymer of the present invention as described in Example 3 by measuring the change in length before and after deformation strain is applied thereto.

FIG. 6A is a photograph of a polymer scaffold made of a lactide/ε-caprolactone polymer scaffold according to the present invention as described in Example 1 immediately after it is transplanted to a cartilage defect area of the rabbit knee joint.

FIG. 6B is a photograph of the cartilage extracted 4 months after a polymer scaffold made of a lactidek-caprolactone polymer scaffold according to the present invention as described in Example 1 is transplanted to a cartilage defect area of the rabbit knee joint.

FIG. 6C is a photograph of the cartilage extracted 4 months after a polymer scaffold made of a lactide/ε-caprolactone polymer scaffold according to the present invention as described in Example 1 is transplanted to a cartilage defect area of the rabbit knee joint, followed by Safranin O staining.

The patent or application file contains at least one drawing executed in color. Copies of this patent or patent application publication with color drawing(s) will be provided by the Office upon request and payment of the necessary fee.

### DETAILED DESCRIPTION OF THE INVENTION

The present invention provides a highly resilient (lactide/glycolide)/ε-caprolactonc copolymer with great shape recovery force, flexibility, and biodegradability.

Cartilage is the portion where various mechanical forces created during normal activities of daily living are inflicted upon, and such mechanical forces play an important role in the cartilage regeneration and chondrogenesis. Natural cartilage exhibits high elasticity, i.e., complete restoration to its original state when mechanical forces imposed thereon are removed. The highly resilient (lactide/glycolide)/ε-caprolactone copolymer according to the present invention is characterized as exhibiting similar mechanical properties (e.g., high shape recovery force, flexibility) and biodegradability, as those of natural cartilage. Such mechanical properties of the (lactide/glycolide)/ε-caprolactone copolymer according to the present invention can be regulated by controlling the molar ratio among the three monomers, lactide, glycolide, and ε-caprolactone.

In particular, the highly resilient (lactide/glycolide)/ε-caprolactone copolymer of the present invention is a biodegradable and biocompatible polymer scaffold having the following properties. First, when tissue cells are seeded on the scaffold, the tissue cells arc easily adhered to and proliferate on the scaffold. In addition, the physiological functions of the differentiated cells are well conserved in the scaffold. After in vivo transplantation, the scaffold is highly compatible with the surrounding tissues and does not cause any inflammatory response. Further, the scaffold is spontaneously degraded by endogeneous enzymes and moisture after a certain period of time.

In one embodiment of the present invention, the highly resilient (lactide/glycolide)/ε-caprolactone copolymer of the present invention may have a weight-average molecular weight (M_{w}) in the range of 10,000 to 500,000. If the weight-average molecular weight of the copolymer is not more than 10,000, the mechanical strength of the copolymer is too weak to use as a polymer scaffold. If weight-average molecular weight of the copolymer exceeds 500,000, it is impossible to achieve high elasticity suitable for cartilage regeneration and it takes a long time for in vivo degradation.

In another embodiment of the present invention, the highly resilient (lactide/glycolide)/ε-caprolactone copolymer of the present invention may have a molar ratio between lactide/glycolide and ε-caprolactone in the range of 65:35 to 35:65. If the molar ratio of lactide/glycolide to ε-caprolactone exceeds 65%, the copolymer would exhibit an excessively high modulus, where it would be too stiff to use as a polymer scaffold. If the molar ratio of lactide/glycolide to ε-caprolactone is not more than 35%, the copolymer would be too soft to use as a polymer scaffold, resulting in the collapse of the porous structure of the scaffold. Further, in terms of the shape recovery force, if the molar ratio of c-caprolactone to lactide/glycolide is not more than 35% or exceeds 65%, when the polymer scaffold is deformed at a strain rate of 300% or higher, it exhibits a low shape recovery force not exceeding 70%. Therefore, in order to use the highly resilient (lactide/glycolide)/ε-caprolactone copolymer of the present invention as a polymer scaffold for the repair of cartilage defects, the molar ratio of ε-caprolactone to lactide/glycolide may be maintained in the range of 35% to 65%, which results in providing a shape recovery force of 70% or greater against deformation at a strain rate of 300% or higher.

Further, the highly resilient (lactide/glycolide)/ε-caprolactone copolymer of the present invention can regulate the in vivo degradation rate where mechanical strength similar to natural cartilage is retained by controlling the molar ratio between lactide and glycolide. While, the higher the molar ratio of glycolide to lactide is, the faster the in vivo degradation rate of the copolymer is, the higher the molar ratio of lactide to glycolide is, the slower its in vivo degradation rate is. In one embodiment of the present invention, the molar ratio between lactide and glycolide may be in the range of 0:10 to 10:0. When the molar ratio for lactide is 0%, the resulting copolymer becomes a glycolide/ε-caprolactone copolymer, while when the molar ratio for glycolide is 0%, the resulting copolymer becomes a lactide/ε-caprolactone copolymer. Further, when the molar ratios for both lactide and glycolide are not 0%, the resulting copolymer becomes a lactide/glycolide/s-caprolactone copolymer. Therefore, the polymer scaffold of the present invention may be prepared by using one of lactide/ε-caprolactonc copolymers, glycolide/ε-caprolactone copolymers, and lactide/glycolide/ε-caprolactone copolymers. The polymer scaffold of the present invention prepared by using the (lactide/glycolide)/ε-caprolactone copolymers having a proper molar ratio of lactide, glycolide, and ε-caprolactone as described above may have a degradation time in the range of 3 months to 3 years.

Generally, polymer scaffolds for tissue engineering should have pores of uniform size, a highly interconnective porous structure, and a certain level of mechanical strength. In one embodiment of the present invention, the highly resilient (lactidc/glycolide)/ε-caprolactone copolymer of the present invention may have a pore size in the range of 1 to 800 μm, which is favorable for functioning as a polymer scaffold. If the pore size is not larger than 1 μm, the pore interconnectivity within the polymer scaffold becomes poor, while if the pore size exceeds 800 μm, there is a problem in terms of a reduction in mechanical strength. Considering both the pore morphology and the mechanical strength of a polymer scaffold, the highly resilient copolymer of the present invention may have a pore size in the range of 30 μm to 800 m, more specifically 50 μm to 500 μm.

In another embodiment of the present invention, the highly resilient (lactide/glycolide)/ε-caprolactone copolymer of the present invention may have a porosity in the range of 40 to 97% so as to function as a polymer scaffold. If the porosity is not more than 40%, the pore interconnectivity within the polymer scaffold is significantly reduced, while if the porosity exceeds 97%, there is a problem in terms of a reduction in mechanical strength. Considering both the pore morphology and the mechanical strength of a polymer scaffold, the highly resilient copolymer of the present invention may have a porosity in the range of 50 to 97%, more specifically 70 to 95%.

The polymer scaffold of the present invention can be prepared from the highly resilient (lactide/glycolide)/ε-caprolactone copolymer by using conventional methods in the art such as, for example, solvent casting, particle leaching, gas foaming, phase separation, electrospinning, gel spinning and the like. The thus prepared polymer scaffold of the present invention can exhibit similar mechanical properties to natural cartilage, such as high elasticity, shape recovery force and flexibility, due to the intrinsic characteristics of the copolymer used. The mechanical strength in connection with elasticity can be assessed according to an elastic recovery test which measures the restoration rate showing the degree of shape recovery from deformation, 5 minutes after a polymer scaffold is deformed under compression or tensile force. In one embodiment of the present invention, the highly resilient (lactide/glycolide)/ε-caprolactone copolymer of the present invention may exhibit a restoration rate of greater than 70%. The restoration rate of the highly resilient copolymer according to the present invention may be varied depending on the pore size and porosity of the copolymer as described above. In order to use the copolymer as a polymer scaffold for cartilage regeneration, the ideal restoration rate of the highly resilient copolymer may be greater than 90%.

Due to the above-mentioned mechanical properties, the (lactide/glycolide)/ε-caprolactone copolymer of the present invention can be easily deformed, e.g., bent, folded, curved, twisted and the like, and exhibits good shape recovery force where it is almost completely restorable to its original form after the deformation strain is removed. Such a good shape recovery force makes it possible to perform an easy transplantation of a polymer scaffold made of the highly resilient copolymer according to the present invention by using an arthroscope. In one embodiment of the present invention, the transplantation may be conducted by folding the polymer scaffold, inserting the folded polymer scaffold into an arthroscope, inserting the arthroscope into a cartilage defect area, pulling out the folded polymer scaffold from the arthroscope, allowing the folded polymer scaffold to be restored to its original form, and then anchoring the polymer scaffold in the original form to the cartilage defect area. As such, since the highly resilient copolymer of the present invention is highly biodegradable and biocompatible and exhibits similar mechanical properties to natural cartilage such as high shape recovery force and flexibility, it can be effectively used as a polymer scaffold for cartilage regeneration and be easily applied to a cartilage defect area by using an arthroscope.

In one embodiment of the present invention, in order to improve the efficiency of cartilage regeneration, the polymer scaffold made of the highly resilient (lactide/glycolide)/ε-caprolactone copolymer according to the present invention may be seeded with cells capable of being differentiated into chondrocytes before the transplantation to thereby form a polymer scaffold-cell composite construct. Suitable examples of cells capable of being differentiated into chondrocytes for the present invention may include mesenchymal stem cells and interstitial cells derived from one of bone marrow, muscle, adipose, umbilical cord, amnion and amniotic fluid; precursor cells derived from the above cells that can be differentiated into chondrocytes; chondrocytes differentiated from the above cells; and primary chondrocytes isolated from cartilage tissue and the like, but are not limited thereto. Such cells can be used alone or as a mixture thereof. The isolation, proliferation, and differentiation into chondrocytes of such cells can be carried out according to conventional methods well known in the art.

The thus prepared polymer scaffold-cell composite construct according to the present invention may be directly transplanted to a cartilage defect area by using an arthroscope. Alternatively, the polymer scaffold-cell composite construct according to the present invention may be cultured in vitro for a certain period of time and then transplanted to a cartilage defect area by using an arthroscope. Any medium for chondrogenic differentiation including DMEM (Dulbecco's modified Eagle's Medium) may be used for the seeding of cells capable of being differentiated into chondrocytes on a polymer scaffold and the in vitro cultivation thereof, so long as it is capable of differentiating such cells into chondrocytes. Considering the efficiency of cartilage regeneration, the cells capable of being differentiated into chondrocytes may be seeded on a polymer scaffold of the present invention at a concentration of 1×10⁵ to 1×10⁸ cells/1 mm′ polymer scaffold.

Since the thus seeded cells on the polymer scaffold of the present invention can receive well the various mechanical stimuli essential for cartilage regeneration through the superior elastic mechanical properties of the polymer scaffold, the cells successfully differentiate into chondrocytes and maintain their chondrocyte phenotype, leading to effective cartilage regeneration.

The polymer scaffold made of the highly resilient (lactide/glycolide)/ε-caprolactone copolymer according to the present invention can be folded alone or in a composite construct with the cells capable of being differentiated into chondrocytes seeded thereon and inserted into an arthroscope. After inserting the arthroscope into a cartilage defect area and pulling out the polymer scaffold from the defect area, the polymer scaffold can be restored to its original form without causing any deformation due to its good shape recovery force, and then, anchored to the cartilage defect area. The polymer scaffold of the present invention has good shape recovery force where it is almost completely restored to its original form when deformation strain applied to the polymer scaffold is removed, which makes it possible to carry out a simple and convenient transplantation of the polymer scaffold using an arthroscope.

Such an arthroscopic transplantation is generally easier and safer on the patient than open surgery and likely to be less physically and mentally traumatic. In case of treating articular cartilage defects by conventional open surgery, the patient must be anesthetized, a cartilage defect area then incised, and a piece of the soft tissue (periosteum) that covers the tibia removed to reveal articular cartilage. Thus, there is the possibility of a scar remaining after the incision and an economic, physical, and mental burden being imposed on the patient due to the anesthesia and long-time operation.

On the other hand, the polymer scaffold made of the highly resilient (lactide/glycolide)/ε-caprolactone copolymer of the present invention exhibits a good shape recovery force of greater than 95% within 5 seconds, even when it is deformed at a strain rate of 500% or higher. Thus, if necessary, after the cells capable of being differentiated into chondrocytes are seeded on the polymer scaffold, a surgeon can roll or fold the resulting polymer scaffold with his or her own hands and insert it into an arthroscope. Next, the arthroscope including the folded polymer scaffold is inserted into a cartilage defect area through a tiny incision, and then, the polymer scaffold is pulled out from the arthroscope and anchored to the defect area. When inserted into the arthroscope, the polymer scaffold of the present invention settles in the arthroscope while adjusting its shape and size, so as to perfectly fit within the inner diameter of the arthroscope and not spontaneously separate from the arthroscope during the transplantation. Thus, the polymer scaffold of the present invention can be transplanted to a target site by using a conventional arthroscope for diagnosis without requiring any special device. After the insertion to a cartilage defect area, the polymer scaffold is separated from the arthroscope and anchored to the defect area by using a conventional adhesive that is commercially available, such as fibrin glue, pins, anchors, screws and the like.

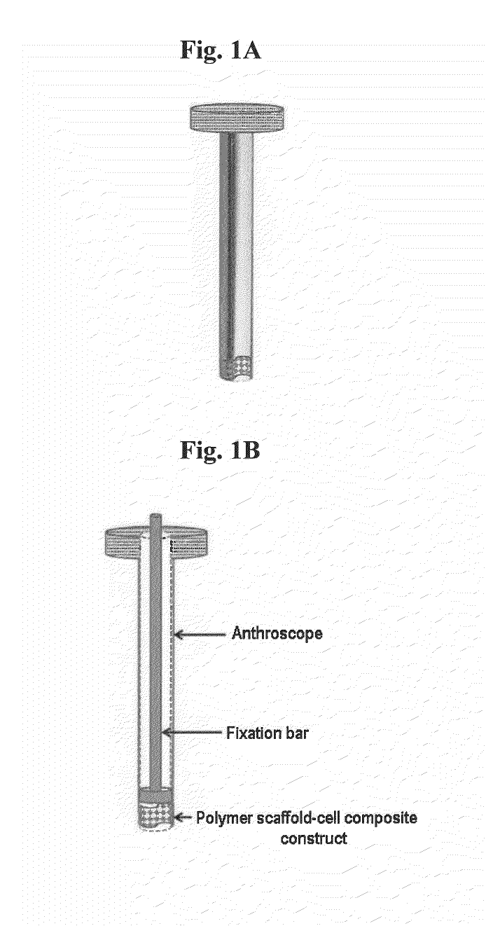

FIG. 1A is a schematic illustration of an arthroscope into which the folded polymer scaffold of the present invention is inserted. In such a case, after the arthroscope including the folded polymer scaffold is inserted into a cartilage defect area and an arthroscopic forceps is inserted into the other side of the defect area, the polymer scaffold is pulled out from the arthroscope by using the arthroscopic forceps, and then, anchored to the defect area. As shown in FIG. 1B, it is possible to manufacture a special arthroscope for the transplantation of the polymer scaffold made of the highly resilient copolymer according to the present invention. FIG. 1B is a schematic illustration of a special manufactured arthroscope into which the folded polymer scaffold of the present invention is inserted, followed by insertion of a fixation bar thereon.

As described above, since the highly resilient (lactide/glycolide)/ε-caprolactone copolymer according to the present invention exhibits high flexibility capable of delivering in vivo mechanical stimuli to seeded cells on the polymer as well as good biodegradability and biocompatibility, it can be effectively used in the repair of cartilage defects. Further, because of the high elasticity and shape recovery force, the highly resilient copolymer of the present invention can be easily deformed, e.g., bent, folded, curved, twisted and the like, and almost completely restored to its original form after the deformation. Such high elasticity and shape recovery force make it possible to fold a polymer scaffold made of the highly resilient copolymer according to the present invention and insert it into an arthroscope. Thus, upon transplantation as a polymer scaffold for the repair of cartilage defects, the coplolymer can be safely and conveniently transplanted to a patient without causing economic, physical and mental burden. Therefore, because of the physical properties favorable to cartilage regeneration and the convenience of the transplantation, the high resilient copolymer of the present invention can be effectively used as a polymer scaffold for the repair of cartilage defects.

### EXAMPLES

Hereinafter, the embodiments of the present invention will be described in more detail with reference to the following examples. However, the examples are only provided for purposes of illustration and are not to be construed as limiting the scope of the invention.

### Example 1

A polymer solution was prepared by dissolving lactidek-caprolactone copolymer (molar ratio of monomers=5:5) having a weight-average molecular weight (M_{w}) of 330,000 in chloroform at a final concentration of 4% (w/v) and homogeneously mixing with a magnetic stirrer. Sodium chloride having an average diameter (φ) of 300 to 500 μm was added to the polymer solution at a final concentration of 85% by weight, based on the total weight of lactide/ε-caprolactone copolymer, and homogeneously mixed with a magnetic stirrer in a hood. The thus obtained mixture of lactide/ε-caprolactone copolymer/sodium chloride/chloroform was exposed to air to evaporate chloroform therefrom until the chloroform was reduced to 25% by weight based on the total weight of the mixture. The resulting mixture was poured into a square tray mold (10×10 cm) and compressed with a oil pressure pump at a pressure of 40 MPa for 3 minutes, to obtain the mixture in the form of a sheet. The mixture in the form of a sheet was kept in a vacuum oven at room temperature for 7 days so as to completely remove the chloroform therefrom. After that, the mixture in the form of a sheet was washed with distilled water nine times for 3 days so as to thoroughly remove the sodium chloride therefrom, followed by freeze-drying, to thereby obtain a polymer scaffold for cartilage regeneration made of the lactide/ε-caprolactone copolymer.

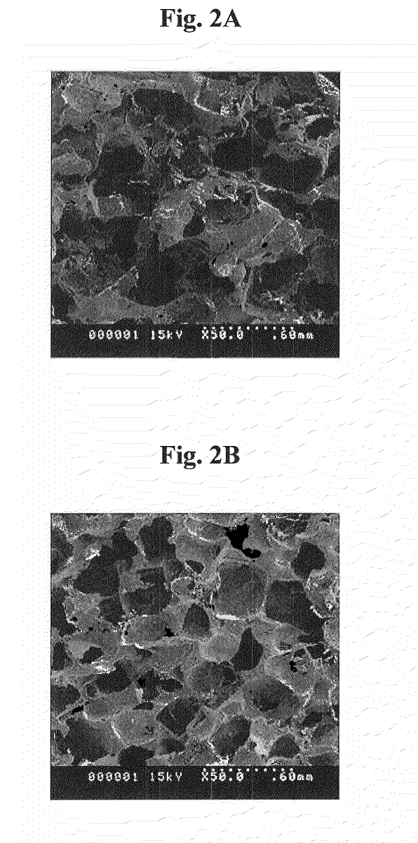

The surface and cross-section of the polymer scaffold obtained above were observed with a scanning electron microscope (SEM), and shown in FIGS. 2A and 2B, respectively. Referring to the results as shown in FIGS. 2A and 2B, it was found that the polymer scaffold made of the highly resilient lactide/ε-caprolactone copolymer according to the present invention showed high interconnectivity between pores and had a uniform pore size.

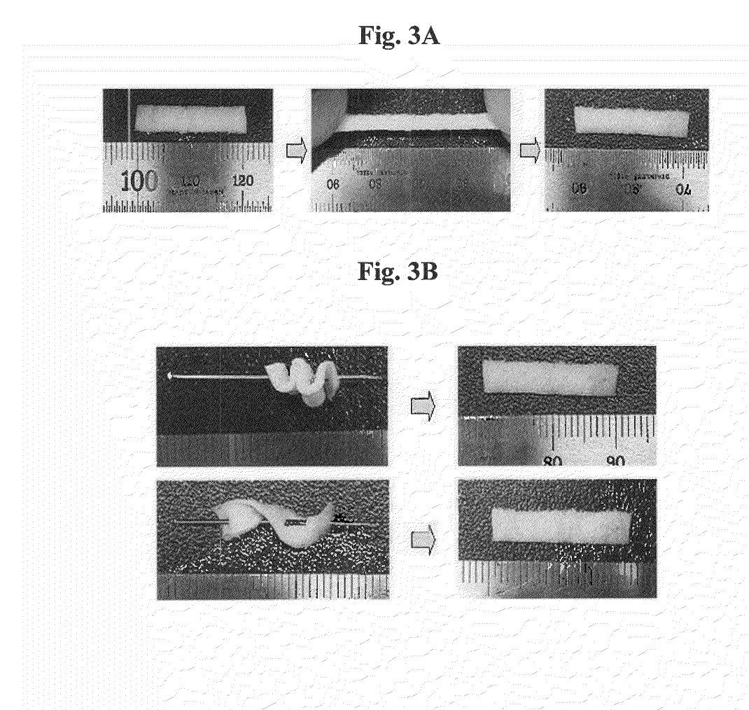

As can be seen from FIG. 3A, it was confirmed that, when the polymer scaffold of the present invention was stretched up to 200% of its original length, it instantaneously restored to its original form. FIG. 3B showed that the polymer scaffold made of the highly resilient lactide/ε-caprolactone copolymer according to the present invention could be bended and folded without any damage to the scaffold.

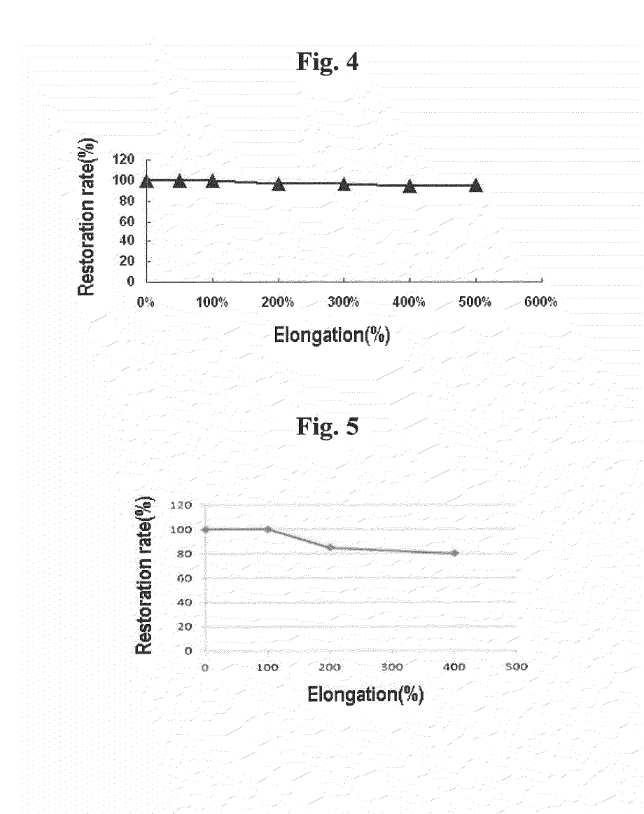

In order to quantitatively analyze the physical properties of the polymer scaffold made of the high resilient lactide/ε-caprolactone copolymer according to the present invention, the following experiment was carried out. The polymer scaffold of the present invention was cut into pieces of approximately 2×1 cm, and the pieces were adhered to a plate while maintaining their length of 1 cm. The pieces were elongated at a rate of 1 cm/min by using a load cell (5 kg) and maintained for 10 seconds in its elongated state. Five minutes after the load cell was removed, the change in length between before and after elongation was measured so as to measure the deformation rate. The deformation rate was calculated using a universal testing machine (Model 5567, Instron Corp., Canton, Mass.). As shown in FIG. 4, it was found that the polymer scaffold made of the highly resilient lactide/ε-caprolactone copolymer according to the present invention showed 95% or higher restoration rate when it was stretched up to 500% of its original length.

### Example 2

A polymer solution was prepared by dissolving glycolide/ε-caprolactone copolymer (molar rate of monomers=5:5) having a weight mean molecular weight (M_{w}) of 104,000 in chloroform at a final concentration of 4% (w/v) and homogeneously mixing with a magnetic stirrer. Sodium chloride having an average diameter (φ) of 300 to 500 μm was added to the polymer solution at a final concentration of 85% by weight, based on the total weight of glycolide/ε-caprolactone copolymer, and homogeneously mixed with a magnetic stirrer in a hood. The thus obtained mixture of glycolide/ε-caprolactone copolymer/sodium chloride/chloroform was exposed to air to evaporate the chloroform, until the chloroform was reduced to 25% by weight based on the total weight of the mixture. The resulting mixture was poured into a square tray mold (10×10 cm) and compressed with a oil pressure pump at a pressure of 40 MPa for 3 minutes, to obtain the mixture in the form of a sheet. The mixture in the form of a sheet was kept in a vacuum oven at room temperature for 7 days so as to completely remove chloroform therefrom. After that, the mixture in the form of a sheet was washed with distilled water nine times for 3 days so as to thoroughly remove sodium chloride therefrom, followed by freeze-drying, to thereby obtain a polymer scaffold for cartilage regeneration made of the glycolidek-caprolactone copolymer.

The surface and cross-section of the polymer scaffold obtained above were observed with a SEM. It was confirmed that the polymer scaffold made of the glycolidek-caprolactone copolymer according to the present invention had a uniform pore size and high pore interconnectivity and showed good elasticity and shape recovery force, similar to the polymer scaffold made of the lactide/ε-caprolactone copolymer in Example 1.

### Example 3

In order to assess the shape recovery force of the (lactide/glycolide)/ε-caprolactone copolymer, a polymer film was prepared by using the same and its shape recovery force was examined, as follows.

A polymer solution was prepared by dissolving lactide/glycolide/ε-caprolactone copolymer (molar rate of monomers=2:3:5) having a weight-average molecular weight (M_{w}) of 184,000 in chloroform at a final concentration of 10% (w/v) and homogeneously mixing with a magnetic stirrer. The polymer solution prepared above was poured onto a glass plate coated with a Teflon film, followed by solvent evaporation, to thereby prepare a polymer film. The thus prepared polymer film was cut into pieces of approximately 2×0.5 an, and the pieces were adhered to a plate while maintaining their length of 0.5 cm. The pieces were elongated at a rate of 1 cm/min by using a load cell (5 kg) and maintained for 10 seconds in its elongated state. Five minutes after the load cell was removed, the change in length between before and after elongation was measured so as to calculate the deformation rate. The deformation rate vias calculated using a universal testing machine (Model 5567, Instron Corp., Canton, Mass.).

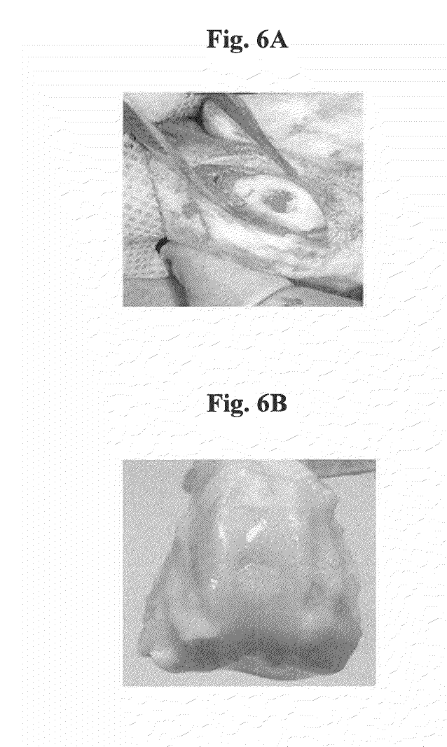

As shown in FIG. 5, it was found that the polymer scaffold made of the highly resilient lactide/glycolide/ε-caprolactone copolymer showed 80% or higher restoration rate when it was stretched up to 400% of its original length.

### Example 4

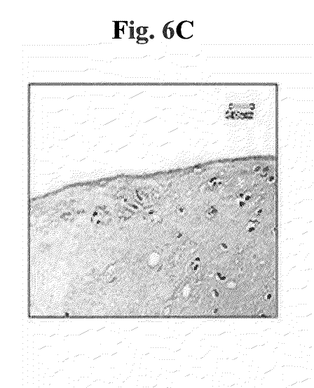

The polymer scaffold made of the highly resilient lactide/ε-caprolactone copolymer of the present invention in Example 1 was transplanted into a cartilage defect area of the rabbit knee joint. In particular, after a 8-week old New Zealand white rabbit was anesthetized, the rabbit knee joint was incised longitudinally (approximately 6 mm in diameter), and the polymer scaffold of the present invention was transplanted thereto. Such a transplantation was to assess whether the physical properties of the polymer scaffold according to the present invention could efficiently induce in vivo cartilage regeneration. FIG. 6A shows the cartilage defect area of the rabbit knee joint immediately after the transplantation of the polymer scaffold according to the present invention. Then, the cartilage defect area of the rabbit knee joint to which the polymer scaffold of the present invention was transplanted was sutured. Four months after, the rabbit was sacrificed and the joint cartilage was extracted. The thus extracted joint cartilage was observed with a naked eye and subjected to a histological analysis. FIG. 6B shows the joint cartilage extracted from the rabbit, 4 months after the polymer scaffold of the present invention was transplanted to the cartilage defect area of the rabbit knee joint. As shown in FIG. 6B, it was found that the polymer scaffold was smoothly connected to the surrounding tissues at the transplanted area, and the cartilage defects were successfully repaired through cartilage regeneration. Histological analysis also showed that new tissues similar to natural cartilage were regenerated at the cartilage defect area. As shown in FIG. 6C, as a result of Safranin O staining, red stained portions within the cartilage defect area to which the polymer scaffold of the present invention was transplanted were detected, suggesting that cartilage-specific matrix molecules similar to natural cartilage were successfully generated.

While the present invention has been described and illustrated with respect to a number of embodiments of the invention, it will be apparent to those skilled in the art that variations and modifications are possible without deviating from the broad principles and teachings of the present invention, which is defined by the claims appended hereto.

What is claimed:

1. A highly resilient copolymer of lactide/glycolide and ε-caprolactone with good shape recovery force, flexibility, and biodegradability.

2. The highly resilient copolymer according to claim 1, which has a weight-average molecular weight (M_{w}) in the range of 10,000 to 500,000.

3. The highly resilient copolymer according to claim 1, wherein the molar ratio between lactide/glycolide and ε-caprolactonc is in the range of 65:35 to 35:65.

4. The highly resilient copolymer according to claim 1, wherein the molar ratio between lactide and glycolide is in the range of 0:10 to 10:0.

5. The highly resilient copolymer according to claim 4, wherein the molar value for lactide is 0 and the copolymer is a glycolide/ε-caprolactone copolymer.

6. The highly resilient copolymer according to claim 4, wherein the molar value for glycolide is 0 and the copolymer is a lactide/ε-caprolactone copolymer.

7. The highly resilient copolymer according to claim 4, wherein the molar value for neither lactide nor glycolide is 0 and the copolymer is a lactide/glycolide/ε-caprolactone copolymer.

8. The highly resilient copolymer according to claim 1, which has a pore size in the range of 1 to 800 μm.

9. The highly resilient copolymer according to claim 1, which has porosity in the range of 40 to 97%.

10. The highly resilient copolymer according to claim 1, which exhibits a shape recovery force of 70% or greater against deformation at a strain rate of 300% or more.

11. A polymer scaffold for the repair of articular cartilage defects comprising the highly resilient copolymer of lactide/glycolide and ε-caprolactone according to claim 1.

12. The polymer scaffold according to claim 11, which is prepared according to a method selected from the group consisting of solvent casting, particle leaching, gas foaming, phase separation, electrospinning, and gel spinning.

13. The polymer scaffold according to claim 11, which is prepared in the form of a cell composite construct by seeding cells capable of being differentiated into chondrocytes on the polymer scaffold.

14. The polymer scaffold according to claim 13, wherein the cells capable of being differentiated into chondrocytes are selected from the group consisting of mesenchymal stem cells and interstitial cells derived from any one of bone marrow, muscle, adipose, umbilical cord, amnion and amniotic fluid; precursor cells derived from said cells that can be differentiated into chondrocytes; chondrocytes differentiated from said cells; primary chondrocytes isolated from cartilage tissue; and mixtures thereof.

15. The polymer scaffold according to claim 13, wherein the cells capable of being differentiated into chondrocytes are seeded at a concentration of 1×10⁵ to 1×10⁸ cells/1 mm polymer scaffold.

16. A method of transplanting the polymer scaffold according to claim 11 to a cartilage defect area by using an arthroscope comprising:
folding a polymer scaffold and inserting it into an arthroscope in a folded state;
inserting the arthroscope into a cartilage defect area
pulling out the folded polymer scaffold from the arthroscope at the cartilage defect area;
allowing the folded polymer scaffold to be restored to its original form; and
anchoring the polymer scaffold to the cartilage defect area.
